(19) United States
(12) Plant Patent Application Publication
(10) Pub. No.: US 2014/0189913 P1
(43) Pub. Date: Jul. 3, 2014

(54) Corylus plant named 'YORK'

(50) ;

(71) Applicant: State of Oregon acting by and through the State Board of Higher Education on behalf of Oregon State University, (US)

(72) Inventors: Shawn A. Mehlenbacher, Corvallis, OR (US); David C. Smith, Corvallis, OR (US); Rebecca L. McCluskey, Corvallis, OR (US)

(73) Assignees: State University, Corvallis, OR (US); State of Oregon acting by and through the State Board of Higher Education of behalf of Oregon

(21) Appl. No.: 13/694,677

(22) Filed: Dec. 24, 2012

(51) Int. Cl. A01H 5/00

(52) U.S. Cl. PLT/152

(57) ABSTRACT

A new and distinct cultivar of Corylus plant named ‘York’ characterized by globose plant habit and moderate vigor, green developing and fully expanded leaves during the spring and summer, resistance to eastern filbert blight caused by the fungus Anisogramma anomala (Peck) E. Müller, presence of random amplified polymorphic DNA markers 152-800 and AA12-850, expression of incompatibility alleles S₂ and S₂₁ in the styles, and DNA fingerprints at 10 of 23 microsatellite marker loci that differ from both parents OSU 479.027 and OSU 504.065, and from one parent at an additional 11 marker loci.

### ACKNOWLEDGMENT OF GOVERNMENT SUPPORT

This invention was made with government support under Specific Cooperative Agreement No. 58-5358-4542 awarded by the United States Department of Agriculture. The government has certain rights in the invention.

### BOTANICAL DENOMINATION

Corylus avellana

### VARIETY DESIGNATION

‘York’

### BACKGROUND

The present Invention relates to a new and distinct cultivar of Corylus plant (hazelnut, filbert), botanically known as Corylus avellana, and hereinafter referred to by the name ‘York’. Corylus avellana is in the family Betulaceae.

The new Corylus resulted from a controlled cross of female parent OSU 479.027 (unpatented) and male parent OSU 504.065 (unpatented) made in 1997 by Shawn A. Mehlenbacher and David C. Smith. Hybrid seeds from the cross were harvested in August 1997, stratified, and seedlings grown in the greenhouse during the summer of 1998. From this cross, total of 183 seedling trees were planted in the field in Corvallis, Oreg., USA in October, 1998. ‘York’ was discovered and selected by the Inventors as a single plant within the progeny of the stated cross-pollination in a controlled environment in Corvallis, Oreg.

‘York’ was originally assigned the designation OSU 878.048, which indicates the row and tree location of the original seedling. OSU 479.027 is from a cross of OSU 231.111 (unpatented)×OSU 226.122 (unpatented). OSU 504.065 is from a cross of OSU 186.080 (unpatented)×VR 17-15 (unpatented). The pedigree of ‘York’ includes ‘Barcelona’ (unpatented) widely grown in Oregon, ‘Casina’ from Asturias, Spain (unpatented), ‘Tonda Gentile delle Langhe’ from Piemonte, northern Italy (unpatented), ‘Montebello’ from Sicily, Italy (unpatented), and ‘Tombul Ghiaghli’, a Turkish cultivar obtained from Greece (unpatented).

The new cultivar was asexually reproduced by rooted suckers annually for eight years (2003-2010) in Corvallis, Oreg. The new cultivar was also asexually propagated by whip grafting in Corvallis, Oreg. The unique features of this new Corylus are stable and reproduced true-to-type in successive generations of asexual reproduction.

### SUMMARY

The following traits have been repeatedly observed and are determined to be the unique characteristics of ‘York’. These characteristics in combination distinguish ‘York’ as a new and distinct cultivar:
1. Globose plant habit and moderate vigor.
2. Green developing and fully expanded leaves during the spring and summer.
3. Resistance to eastern filbert blight caused by the fungus Anisogramma anomala (Peck) E. Müller.
4. Presence of random amplified polymorphic DNA markers 152-800 and AAl12-850 in DNA of ‘York’ amplified by the polymerase chain reaction. These two markers are linked to a dominant allele for resistance to eastern filbert blight from the cultivar Gasaway (unpatented).
5. Expression of incompatibility alleles S₂ and S₂₁ in the styles.
6. DNA fingerprints at 10 of 23 microsatellite marker loci differ from both parents OSU 479.027 and OSU 504.065, and from one parent at an additional 11 marker loci. The microsatellite primers are shown in Table 1, and allele sizes are shown in Table 2. DNA fingerprints of standard cultivars ‘Barcelona’, ‘Tonda Gentile delle Langhe’ and ‘Extra Ghiaghli’, and ‘Gasaway’, the source of eastern filbert blight resistance, are also shown in attached Table 2.

In comparisons in two replicated trials conducted in Corvallis, Oreg., plants of the new Corylus differed from plants of the Corylus avellana cultivar Barcelona (unpatented), and other cultivars and selections of Corylus avellana known to the Inventors primarily in nut size, nut shape, kernel percentage (ratio of kernel weight to nut weight), frequency of blank nuts (nuts lacking kernels), time of pollen shed, time of nut maturity, length of the husk or involucre, and plant size.

### BRIEF DESCRIPTION OF THE DRAWING

The accompanying colored photographs illustrate the overall appearance of the new cultivar, showing the colors as true as it is reasonably possible to obtain in colored reproductions of this type. Foliage colors in the photographs may differ slightly from the color values cited in the detailed botanical description which accurately describe the colors of the new Corylus.

FIG. 1 shows a tree of the new cultivar ‘York’ growing in a field in August, in Corvallis, Oreg.

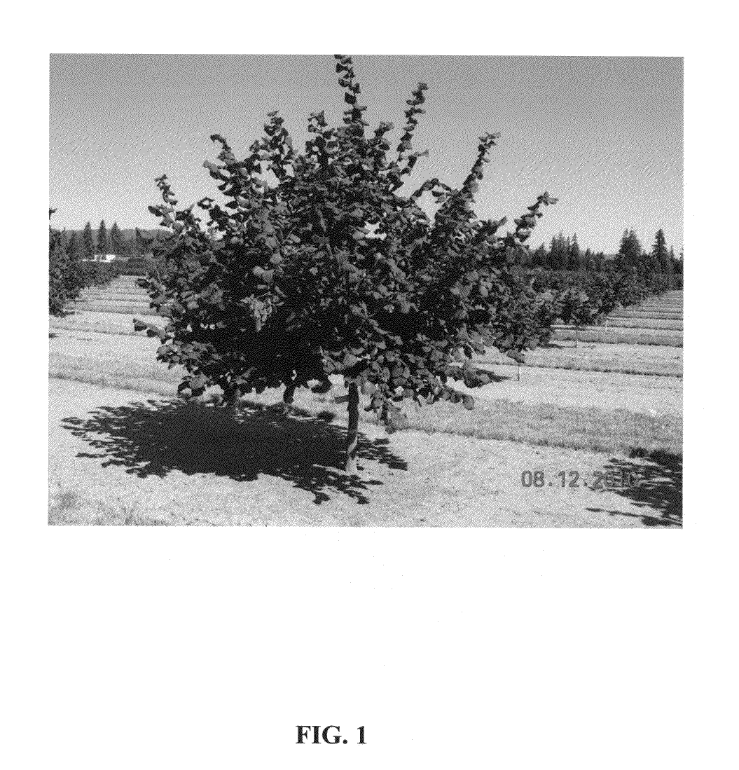

FIG. 2 shows the tree of the new cultivar ‘York’ growing in a field in January, in Corvallis, Oreg.

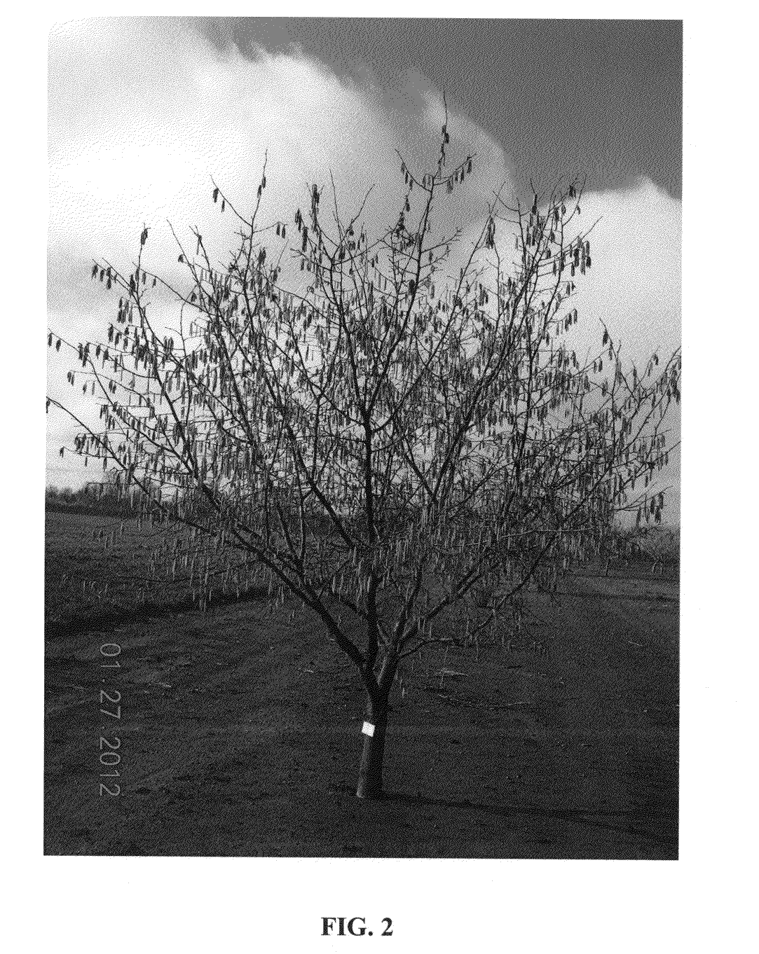

FIG. 3 shows typical nuts, raw kernels, and blanched kernels of ‘York’ hazelnut compared to those of ‘Jefferson’ hazelnut.

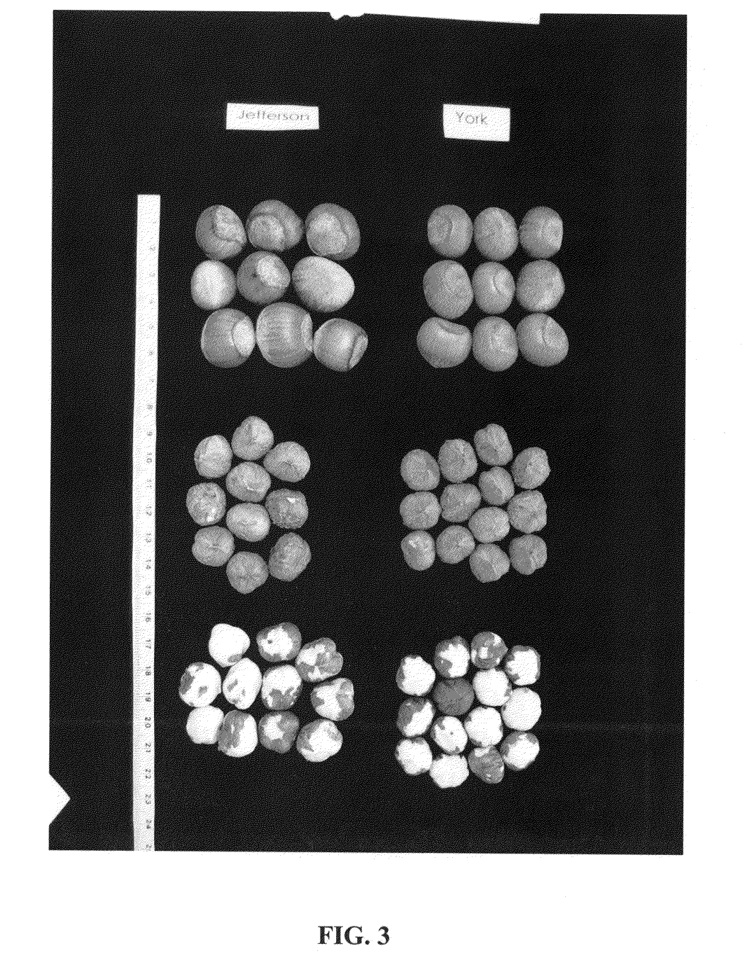

FIG. 4 shows typical nuts, raw kernels, and blanched kernels of ‘York’ hazelnut compared to those of ‘Lewis’ hazelnut.

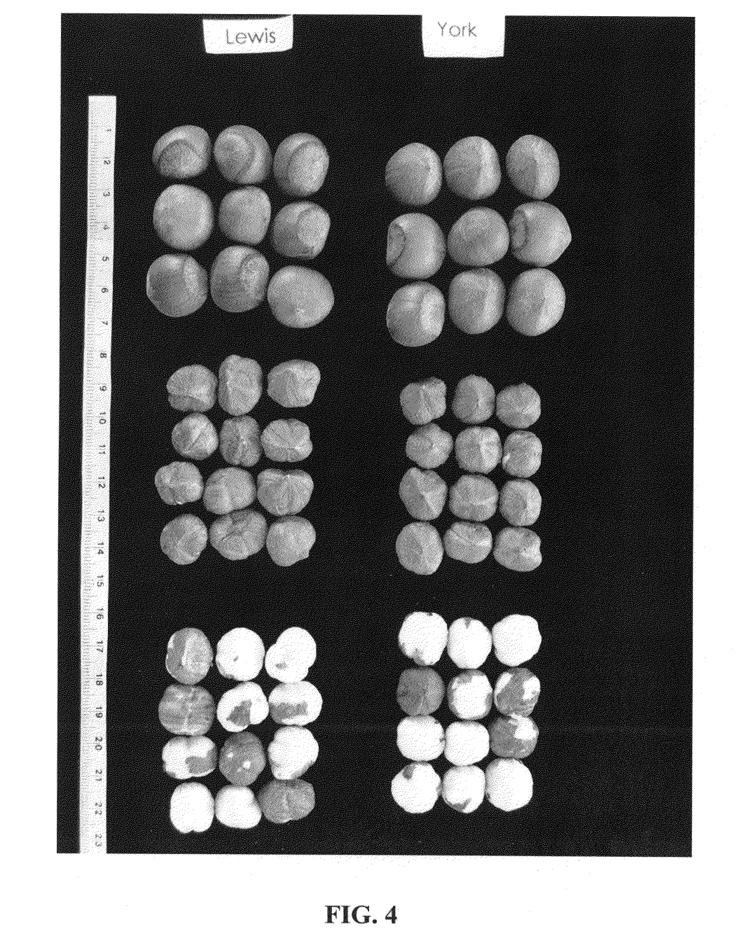

FIG. 5 shows the typical nuts, raw kernels, and blanched kernels of ‘York’ hazelnut compared to those of ‘Barcelona’ hazelnut and other hazelnut cultivars.

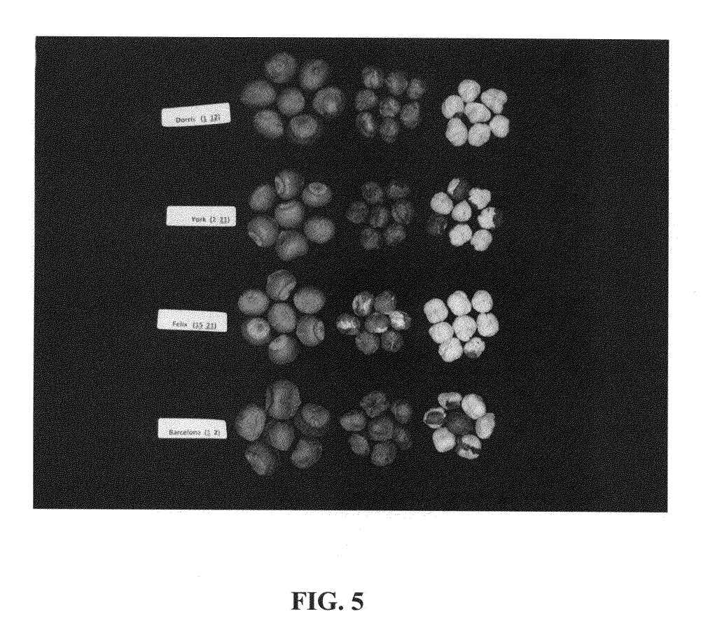

### DETAILED BOTANICAL DESCRIPTION

The cultivar York has not been observed under all possible environmental conditions. The phenotype may vary somewhat with variations in environment such as temperature and light intensity, without, however, any variance in genotype. The aforementioned photographs and following observations and measurements describe plants grown in Corvallis, Oreg. under commercial practice outdoors in the field during the fall, winter and spring. Plants used for the photographs and description were propagated by tie-off layerage and growing on their own roots, and about seven years old. In the following description, color references are made to The Royal Horticultural Society Colour Chart, 1966 Edition, except where general terms of ordinary dictionary significance are used.
Botanical classification: Corylus avellana cultivar York.
Parentage:
   Female, or seed, parent.—Corylus avellana selection OSU 479.027 (unpatented).
   Male, or pollen, parent.—Corylus avellana selection OSU 504.065 (unpatented).
Propagation (type rooted suckers):
   Time to initiate roots.—about 30 days at 20 degrees C.
   Time to produce a rooted young plant.—about six months at 22 degrees C.
   Root description.—fine to thick; freely branching; creamy white in color.
Propagation (type whip grafting):
   Time to budbreak on the scions.—about 14 days at 25° C.
   Time to produce a grafted plant.—about six months at 25° C.
Plant description:
   General appearance.—perennial shrub.
   Size.—plant height is about 5 meters; plant diameter or spread is about 5 meters.
   Form.—spreading plant habit.
   Growth and branching habit.—freely branching; about 15 lateral branches develop per plant. Pinching, i.e., removal of the terminal apices, enhances branching with lateral branches potentially forming at every node.
   Vigor.—Moderate vigor growth habit.
   Roots.—Fine to thick; freely branching; creamy white in color.
Lateral branch description:
   Length.—about 38 cm.
   Diameter.—about 6 mm.
   Internode length.—about 3.2 cm.
   Texture.—smooth, glabrous.
   Strength.—strong.
   Color.—immature — 152B, mature — 152B.
Foliage description:
   Arrangement.—alternate, simple.
   Length.—about 10.0 cm.
   Width.—about 9.3 cm.
   Shape.—ablong to ovate.
   Apex.—obtuse to acute.
   Base.—cordate.
   Margin.—serrate.
   Texture.—upper and lower surfaces — slightly pubescent.
   Venation pattern.—pinnate.
   Color.—Developing foliage: upper surface 146B, lower surfaces: 146C. Fully expanded foliage, upper surface: Spring and summer, 146A; late summer and fall, 146A. Fully expanded foliage, lower surface: Spring and summer, 146C; late summer and fall, 146C. Venation, upper surface: Spring and summer, 146A; late summer and fall, 146A. Venation, lower surface: Spring and summer, 148D; late summer and fall, 148D.
Petiole description:
   Length.—about 2.7 cm.
   Diameter.—about 1.8 mm.
   Texture.—upper and lower surfaces: pubescent.
   Color.—upper surface: Spring and summer, 139D; late summer and fall, 139D. lower surface: Spring and summer, 139D; late summer and fall, 139D.
Flower description:
   Male inflorescences.—catkins, color prior to elongation 194C.
   Female inflorescence.—style color 047B.
Nut description:
   Length.—about 18.0 mm.
   Width.—about 19.7 mm.
   Depth.—about 17.0 mm.
   Nut shape.—round.
   Nut shape index [(width+depth)/2*length].—1.02.
   Nut compression index (width/depth).—1.16.
   Nut shell color.—164A.
   Nut weight.—about 2.73 grams.
   Kernel weight.—about 1.23 grams.
   Kernel percentage (kernel weight/nut weight).—about 46%.
Disease/pest resistance: Plants of the new Corylus are highly resistant to eastern filbert blight caused by the fungus Anisogramma anomala (Peck) E. Müller. Plants of the new Corylus are highly resistant to bud mites (Phytoptus avellanae Nal.), while plants of ‘Tonda Gentile delle Langhe’ are highly susceptible, and plants of ‘Barcelona’ are highly resistant.
Temperature tolerance: Tolerates temperatures from −10 to 38° C. in the field in Corvallis, Oreg.

TABLE 1
Primers and annealing temperatures for the
24 microsatellite marker loci
used to fingerprint
‘York’ and other hazelnut cultivars.

| | Repeat | | | | | | | |
|---|---|---|---|---|---|---|---|---|
| Locus | motif | Size | Tₙ | n | He | Ho | PIC | r |
| A613 | (TC)₁₃ | 149- | 60 | 14 | 0.85 | 0.85 | 0.85 | 0.00 |
| | (CA)₁₂ | 177 | | | | | | |
| A614 | (TC)₁₇ | 125- | 60 | 14 | 0.85 | 0.85 | 0.84 | 0.00 |
| | (CA)₁₀ | 156 | | | | | | |
| | NNN | | | | | | | |
| | (CA)₆ | | | | | | | |
| A616 | (AC)₁₁ | 136- | 60 | 13 | 0.85 | 0.85 | 0.83 | 0.00 |
| | | 162 | | | | | | |
| A640 | (CT)₁₅ | 354- | 67 | 11 | 0.80 | 0.73 | 0.7 | 0.04 |
| | (CA)₁₃ | 378 | | | | | | |
| B107 | (CT)₁₄ | 112- | 55 | 14 | 0.85 | 0.80 | 0.83 | 0.02 |
| | | 151 | | | | | | |
| B617 | (GA)₁₅ | 280- | 60 | 9 | 0.80 | 0.78 | 0.78 | 0.01 |
| | | 298 | | | | | | |
| B619 | (TC)₂₁ | 146- | 60 | 14 | 0.88 | 0.88 | 0.7 | 0.00 |
| | | 180 | | | | | | |
| B634 | (AG)₁₅ | 218- | 60 | 9 | 0.76 | 0.76 | 0.73 | 0.00 |
| | | 238 | | | | | | |
| B657 | (AG)₁₅ | 210- | 60 | 8 | 0.84 | 0.98 | 0.82 | −0.08 |
| | | 228 | | | | | | |
| B671 | (AG) | 221- | 60 | 13 | 0.86 | 0.88 | 0.84 | −0.01 |
| | 6NN | 249 | | | | | | |
| | (GA)₁₇ | | | | | | | |
| B709 | (GA)₂₁ | 219- | 60 | 8 | 0.74 | 0.76 | 0.70 | −0.01 |
| | | 233 | | | | | | |
| B733 | (TC)₁₅ | 161- | 60 | 8 | 0.68 | 0.68 | 0.63 | 0.00 |
| | | 183 | | | | | | |
| B741 | (GT)₅ | 176- | 60 | 10 | 0.77 | 0.78 | 0.74 | 0.00 |
| | (GA)₁₂ | 194 | | | | | | |
| B749 | (TC)₁₂ | 200- | 60 | 6 | 0.60 | 0.64 | 0.51 | −0.03 |
| | | 210 | | | | | | |
| B751 | (GA)₁₅ | 141- | 60 | 7 | 0.80 | 0.80 | 0.77 | 0.01 |
| | | 153 | | | | | | |
| B774 | (AG)₁₅ | 195- | 60 | 8 | 0.80 | 0.80 | 0.77 | 0.01 |
| | | 213 | | | | | | |
| B776 | (GA)₁₇ | 134- | 60 | 7 | 0.71 | 0.60 | 0.67 | 0.07 |
| | | 148 | | | | | | |
| B795 | (TC)₈ | 296- | 60 | 12 | 0.76 | 0.74 | 0.74 | 0.01 |
| | Ns | 332 | | | | | | |
| | (CT)₇ | | | | | | | |
| | Ns(CT) | | | | | | | |
| | ₁₀Ns | | | | | | | |
| | (TC)₅ | | | | | | | |
| C115 | (TAA)₅ | 167- | 60 | 14 | 0.80 | 0.80 | 0.77 | 0.00 |
| | (GAA)₁₂ | 226 | | | | | | |
| KG809 | (AGG)₆ | 333- | 55 | 5 | 0.66 | 0.64 | 0.60 | 0.01 |
| | | 345 | | | | | | |
| KG811 | (GA)₁₇ | 240- | 58 | 12 | 0.83 | 0.82 | 0.81 | 0.01 |
| | | 278 | | | | | | |
| KG827 | (CT)₁₃ | 264- | 67 | 9 | 0.78 | 0.84 | 0.75 | −0.04 |
| | AA | 282 | | | | | | |
| | (CA)₇ | | | | | | | |
| KG830 | (CT)₁₄ | 279- | 67 | 9 | 0.79 | 0.78 | 0.76 | 0.00 |
| | GTATT | 311 | | | | | | |
| | (CA)₈ | | | | | | | |
| Soman- | (AAT)₅ | | 54 | 3 | 0.60 | 0.98 | 0.51 | −0.27 |
| G | | | | | | | | |

| | | Primers | Primers | Refer- |
|---|---|---|---|---|
| Locus | LG | 5′-3′ | 5′-3′ | ence |
| A613 | 11 | Ned- | R- | Gurcan |
| | | CACACG | CCCCTT | et al. |
| | | CCTTGTCA | TCACATGT | 2010 |
| | | CTCTTT | TTGCTT | |
| | | (SEQ ID | (SEQ ID | |
| | | NO: 1) | NO: 2) | |
| A614 | 6 | Hex- | R- | Gurcan |
| | | TGGCAGA | GCAGTGG | et al. |
| | | GCTTTGT | AGGATTG | 2010 |
| | | CAGCTT | CTGACT | |
| | | (SEQ ID | (SEQ ID | |
| | | NO: 3) | NO: 4) | |
| A616 | 8 | Fam- | R- | Gurcan |
| | | CACTCAT | ATGGCTT | et al. |
| | | ACCGCAA | TTGCTTC | 2010 |
| | | ACTCCA | GTTTTG | |
| | | (SEQ ID | (SEQ ID | |
| | | NO: 5) | NO: 6) | |
| A640 | 10 | F- | Fam- | Gurcan |
| | | TGCCTCT | CGCCATAT | et al. |
| | | GCAGTTA | AATTGGGAT | 2010 |
| | | GTCATCAA | GCTTGTTG | |
| | | ATGTAGG | (SEQ ID | |
| | | (SEQ ID | NO: 8) | |
| | | NO: 7) | | |
| B107 | 10 | Ned- | R- | Boccacci |
| | | GTAGGTGC | AACACCATA | et al. |
| | | ACTTGATG | TTGAGTCT | 2005; |
| | | TGCTTTAC | TTCAAAGC | Go- |
| | | (SEQ ID | (SEQ ID | kirmak |
| | | NO: 9) | NO: 10) | et al. |
| | | | | 2009 |
| B617 | 8 | Fam- | R- | Gurcan |
| | | TCCGTGT | TGTTTT | et al. |
| | | TGAGTAT | TGGTGGA | 2010 |
| | | GGACGA | GCGATG | |
| | | (SEQ ID | (SEQ ID | |
| | | NO: 11) | NO: 12) | |
| B619 | 3 | Fam- | R- | Gurcan |
| | | AGTCGG | GCGATC | et al. |
| | | CTCCCCT | TGACCTC | 2010 |
| | | TTTCTC | ATTTTTG | |
| | | (SEQ ID | (SEQ ID | |
| | | NO: 13) | NO: 14) | |
| B634 | 4 | Hex- | R- | Gurcan |
| | | CCTGCA | GTGCAG | et al. |
| | | TCCAGGA | AGGTTGC | 2010 |
| | | CTCATTA | ACTCAAA | |
| | | (SEQ ID | (SEQ ID | |
| | | NO: 15) | NO: 16) | |
| B657 | 11 | Ned- | R- | Gurcan |
| | | GAGAGT | AGCCTC | et al. |
| | | GCGTCTT | CAACCTC | 2010 |
| | | CCTCTGG | ACGAAC | |
| | | (SEQ ID | (SEQ ID | |
| | | NO: 17) | NO: 18) | |
| B671 | 9 | Hex- | R- | Gurcan |
| | | TTGCCAG | ACCAGC | et al. |
| | | TGCATAC | TCTGGGC | 2010 |
| | | TCTGATG | TTAACAC | |
| | | (SEQ ID | (SEQ ID | |
| | | NO: 19) | NO: 20) | |
| B709 | 5 | Ned- | R- | Gurcan |
| | | CCAAGCA | GCGGGT | et al. |
| | | CGAATGA | TCTCGTT | 2010 |
| | | ACTCAA | GTACACT | |
| | | (SEQ ID | (SEQ ID | |
| | | NO: 21) | NO: 22) | |
| B733 | 7.2 | Ned- | R- | Gurcan |
| | | CACCCT | CATCCC | et al. |
| | | CTTCACC | CTGTTGG | 2010 |
| | | ACCTCAT | AGTTTTC | |
| | | (SEQ ID | (SEQ ID | |
| | | NO: 23) | NO: 24) | |
| B741 | 5 | Fam- | R- | Gurcan |
| | | GTTCACA | CGTGTT | et al. |
| | | GGCTGTT | GCTCATG | 2010 |
| | | GGGTTT | TGTTGTG | |
| | | (SEQ ID | (SEQ ID | |
| | | NO: 25) | NO: 26) | |
| B749 | 1 | Hex- | R- | Gurcan |
| | | GGCTGA | TCGGCTA | et al. |
| | | CAACACA | GGGTTAG | 2010 |
| | | GCAGAAA | GGTTTT | |
| | | (SEQ ID | (SEQ ID | |
| | | NO: 27) | NO: 28 | |
| B751 | 7.2 | Fam- | R- | Gurcan |
| | | AGCTGGT | AAACTCAA | et al. |
| | | TCTTCGA | ATAAAACC | 2010 |
| | | CATTCC | CCTGCTC | |
| | | (SEQ ID | (SEQ ID | |
| | | NO: 29) | NO: 30) | |
| B774 | 5 | Ned- | R- | Gurcan |
| | | GTTTTG | TGTGTGT | et al. |
| | | CGAGCTC | GGTCTGT | 2010 |
| | | ATTGTCA | AGGCACT | |
| | | (SEQ ID | (SEQ ID | |
| | | NO: 31) | NO: 32) | |
| B776 | 6 | Fam- | R- | Gurcan |
| | | TGTATGTA | TGAGGGG | et al. |
| | | CACACGGA | AAGAGGT | 2010 |
| | | GAGAGAGA | TTGATG | |
| | | (SEQ ID | (SEQ ID | |
| | | NO: 33) | NO: 34) | |
| B795 | NA | Fam- | R- | Gurcan |
| | | GACCCACA | TGGGCA | et al. |
| | | AACAATAA | TCATCCA | 2010 |
| | | CCTATCTC | GGTCTA | |
| | | (SEQ ID | (SEQ ID | |
| | | NO: 35) | NO: 36) | |
| C115 | 4 | Fam- | GTTTCCAG | Bassil |
| | | A1111 CC | ATCTGCCTC | et al. |
| | | GCAGATA | CATATAAT | 2005b, |
| | | ATACAGG | (SEQ ID | Go- |
| | | (SEQ ID | NO: 38) | kirmak |
| | | NO: 37) | | et al. |
| | | | | 2009 |
| KG809 | 4 | Hex- | F- | Gurcan |
| | | AGGCAT | GGAAGGT | and |
| | | CAGTTC | GAGAGAA | Mehlen- |
| | | ATCCAA | ATCAAGT | bacher |
| | | (SEQ ID | (SEQ ID | 2010 |
| | | NO: 39) | NO: 40) | |
| KG811 | 2 | Ned- | F- | Gurcan |
| | | AAGGCG | GAACAAC | and |
| | | GCACTC | TGAAGAC | Mehlen- |
| | | GCTCACA | GCAAAG | bacher |
| | | (SEQ ID | (SEQ ID | 2010 |
| | | NO: 41) | NO: 42) | |
| KG827 | 9 | Fam- | GAGGGA | Gurcan |
| | | AGAACTCC | GCAAGTCA | and |
| | | GACTAATA | AAGTTGA | Mehlen- |
| | | ATCCTAA | GAAGAAA | bacher |
| | | CCCTTGC | (SEQ ID | 2010 |
| | | (SEQ ID | NO: 44) | |
| | | NO: 43) | | |
| KG830 | 9 | Ned- | AAAGCA | Gurcan |
| | | TGGAGGA | ACTCA | and |
| | | AGTTTTGA | TAGCT | Mehlen- |
| | | ATGGTAG | GAAGTC | bacher |
| | | TAGAGGA | CAATCA | 2010 |
| | | (SEQ ID | (SEQ ID | |
| | | NO: 45) | NO: 46) | |
| Soman-G | NA | Hex- | R- | unpub- |
| | | TGGCGT | GCCATCTTT | lished |
| | | TGCAACA | AGAAAGTTC | |
| | | TATTCTC | GATACAG | |
| | | (SEQ ID | (SEQ ID | |
| | | NO: 47) | NO: 48) | |

Primer fluorescent tags are FAM, HEX, and NED.
Ta: annealing-temperature (° C.)
N: number of alleles
He: expected heterozygosity
Ho: observed heterozygosity
PIC: polymorphism information content
r: estimated null allele frequency
LG: linkage group

TABLE 2
Allele sizes in ‘York’, its parents, and four other hazelnut cultivars
at 24 microsatellite loci.

| | | | | | ‘Tonda Gentile |
|---|---|---|---|---|---|
| Tag | Locus | ‘York’ | 479.027 | 504.065 | delle Langhe’ |
| NED | A613 | 157/177 | 177/177 | 157/159 | 151/157 |
| HEX | A614 | 125/158 | 125/132 | 148/158 | 125/135 |
| FAM | A616 | 142/150 | 142/150 | 150/150 | 148/150 |
| FAM | A640 | 362/374 | 354/374 | 354/362 | 354/368 |
| NED | B107 | 122/134 | 134/146 | 122/134 | 134/152 |
| FAM | B617 | 286/290 | 286/290 | 286/286 | 286/296 |
| FAM | B619 | 156/164 | 156/164 | 156/164 | 148/164 |
| HEX | B634 | 226/234 | 226/226 | 232/234 | 226/226 |
| NED | B657 | 220/222 | 222/226 | 220/224 | 218/226 |
| HEX | B671 | 241/247 | 227/247 | 241/241 | 237/241 |
| NED | B709 | 227/231 | 227/231 | 227/227 | 227/227 |
| NED | B733 | 171/179 | 171/179 | 173/179 | 171/173 |
| FAM | B741 | 177/186 | 177/186 | 177/184 | 177/184 |
| HEX | B749 | 208/208 | 206/208 | 206/208 | 206/208 |
| FAM | B751 | 151/153 | 151/153 | 143/151 | 149/153 |
| NED | B774 | 203/209 | 209/211 | 203/207 | 203/211 |
| FAM | B776 | 137/150 | 137/137 | 137/150 | 137/137 |
| FAM | B795 | 330/330 | 296/330 | 330/330 | 312/330 |
| FAM | C115 | 197/197 | 194/197 | 194/197 | 173/173 |
| HEX | KG809 | 336/345 | 336/339 | 339/345 | 336/339 |
| NED | KG811 | 254/254 | 254/254 | 254/264 | 254/264 |
| FAM | KG827 | 266/270 | 270/282 | 266/266 | 266/268 |
| NED | KG830 | 295/295 | 295/295 | 295/295 | 291/295 |
| HEX | SMNG | 196/200 | 196/200 | 196/200 | 196/200 |

| | | ‘Extra | |
|---|---|---|---|
| Tag | ‘Barcelona’ | Ghiaghli’ | ‘Gasaway’ |
| NED | 151/159 | 167/169 | 159/161 |
| HEX | 125/131 | 125/150 | 143/158 |
| FAM | 142/150 | 150/158 | 148/148 |
| FAM | 354/374 | 374/374 | 362/368 |
| NED | 112/134 | 116/116 | 122/128 |
| FAM | 286/290 | 294/296 | 292/296 |
| FAM | 156/170 | 164/174 | 170/174 |
| HEX | 226/226 | 226/226 | 220/232 |
| NED | 218/222 | 210/222 | 224/228 |
| HEX | 223/227 | 227/247 | 235/247 |
| NED | 225/233 | 225/227 | 227/227 |
| NED | 171/173 | 171/171 | 173/173 |
| FAM | 177/186 | 177/184 | 186/188 |
| HEX | 208/208 | 208/208 | 206/208 |
| FAM | 143/153 | 143/147 | 143/143 |
| NED | 203/207 | 195/203 | 203/209 |
| FAM | 135/137 | 135/137 | 146/150 |
| FAM | 330/330 | 296/310 | 314/316 |
| FAM | 173/194 | 182/194 | 215/218 |
| HEX | 336/336 | 336/339 | 336/345 |
| NED | 258/264 | 240/242 | 254/258 |
| FAM | 280/282 | 276/282 | 270/280 |
| NED | 291/295 | 291/295 | 291/305 |
| HEX | 196/200 | 196/200 | 196/196 |

### REFERENCES

Bassil N. V., Botta R., Mehlenbacher S. A. 2005a. Microsatellite markers in hazelnut: Isolation, characterization and cross-species amplification. J. Amer. Soc. Hort. Sci. 130:543-549.
Bassil N. V., Botta R., Mehlenbacher S. A. 2005b. Additional microsatellite markers of the European hazelnut. Acta Hort. 686:105-110.
Boccacci P., Akkak A., Bassil N. V., Mehlenbacher S. A., Botta R. 2005. Characterization and evaluation of microsatellite loci in European hazelnut (C. avellana) and their transferability to other Corylus species. Molec. Ecol. Notes 5:934-937.
Boccacci P., Akkak, A. and Botta, R. 2006. DNA typing and genetic relations among European hazelnut (Corylus avellana L.) cultivars using microsatellite markers. Genome 49:598-611.
Gökirmak T., Mehlenbacher S. A., Bassil N. V. 2009. Characterization of European hazelnut (Corylus avellana) cultivars using SSR markers. Genetic Resources and Crop Evolution 56:147-172.
Gürcan, K., S. A. Mehlenbacher and V. Erdogan. 2010a. Genetic diversity in hazelnut cultivars from Black Sea countries assessed using SSR markers. Plant Breeding (available on-line doi:10.1111/j.1439-0523.2009.01753.x).
Gürcan, K., S. A. Mehlenbacher, N. V. Bassil, P. Boccacci, A. Akkak and R. Botta. 2010b. New microsatellite markers for Corylus avellana from enriched libraries. Tree Genetics and Genomes (available on-line as DOI 10.1007/s11295-010-0269-y).
Gürcan, K. and S. A. Mehlenbacher. 2010. Development of microsatellite marker loci for European hazelnut (Corylus avellana L.) from ISSR fragments. Molecular

Breeding (available on-line).

We claim:

1. A new and distinct cultivar of Corylus plant named ‘York’, as illustrated and described.


SEQUENCE LISTING (48 sequences)

SEQ ID NO: 1 - DNA, 20 bp, Artificial Sequence
  Synthetic polynucleotide
cacacgccttgtcactcttt

SEQ ID NO: 2 - DNA, 20 bp, Artificial Sequence
  Synthetic polynucleotide
cccctttcacatgtttgctt

SEQ ID NO: 3 - DNA, 20 bp, Artificial Sequence
  Synthetic polynucleotide
tggcagagctttgtcagctt

SEQ ID NO: 4 - DNA, 20 bp, Artificial Sequence
  Synthetic polynucleotide
gcagtggaggattgctgact

SEQ ID NO: 5 - DNA, 20 bp, Artificial Sequence
  Synthetic polynucleotide
cactcataccgcaaactcca

SEQ ID NO: 6 - DNA, 20 bp, Artificial Sequence
  Synthetic polynucleotide
atggcttttgcttcgttttg

SEQ ID NO: 7 - DNA, 29 bp, Artificial Sequence
  Synthetic polynucleotide
tgcctctgcagttagtcatcaaatgtagg

SEQ ID NO: 8 - DNA, 25 bp, Artificial Sequence
  Synthetic polynucleotide
cgccatataattgggatgcttgttg

SEQ ID NO: 9 - DNA, 24 bp, Artificial Sequence
  Synthetic polynucleotide
gtaggtgcacttgatgtgctttac

SEQ ID NO: 10 - DNA, 25 bp, Artificial Sequence
  Synthetic polynucleotide
aacaccatattgagtctttcaaagc

SEQ ID NO: 11 - DNA, 20 bp, Artificial Sequence
  Synthetic polynucleotide
tccgtgttgagtatggacga

SEQ ID NO: 12 - DNA, 19 bp, Artificial Sequence
  Synthetic polynucleotide
tgtttttggtggagcgatg

SEQ ID NO: 13 - DNA, 19 bp, Artificial Sequence
  Synthetic polynucleotide
agtcggctccccttttctc

SEQ ID NO: 14 - DNA, 20 bp, Artificial Sequence
  Synthetic polynucleotide
gcgatctgacctcatttttg

SEQ ID NO: 15 - DNA, 20 bp, Artificial Sequence
  Synthetic polynucleotide
cctgcatccaggactcatta

SEQ ID NO: 16 - DNA, 20 bp, Artificial Sequence
  Synthetic polynucleotide
gtgcagaggttgcactcaaa

SEQ ID NO: 17 - DNA, 20 bp, Artificial Sequence
  Synthetic polynucleotide
gagagtgcgtcttcctctgg

SEQ ID NO: 18 - DNA, 19 bp, Artificial Sequence
  Synthetic polynucleotide
agcctcacctccaacgaac

SEQ ID NO: 19 - DNA, 21 bp, Artificial Sequence
  Synthetic polynucleotide
ttgccagtgcatactctgatg

SEQ ID NO: 20 - DNA, 20 bp, Artificial Sequence
  Synthetic polynucleotide
accagctctgggcttaacac

SEQ ID NO: 21 - DNA, 20 bp, Artificial Sequence
  Synthetic polynucleotide
ccaagcacgaatgaactcaa

SEQ ID NO: 22 - DNA, 20 bp, Artificial Sequence
  Synthetic polynucleotide
gcgggttctcgttgtacact

SEQ ID NO: 23 - DNA, 20 bp, Artificial Sequence
  Synthetic polynucleotide
caccctcttcaccacctcat

SEQ ID NO: 24 - DNA, 20 bp, Artificial Sequence
  Synthetic polynucleotide
catcccctgttggagttttc

SEQ ID NO: 25 - DNA, 20 bp, Artificial Sequence
  Synthetic polynucleotide
gttcacaggctgttgggttt

SEQ ID NO: 26 - DNA, 20 bp, Artificial Sequence
  Synthetic polynucleotide
cgtgttgctcatgtgttgtg

SEQ ID NO: 27 - DNA, 20 bp, Artificial Sequence
  Synthetic polynucleotide
ggctgacaacacagcagaaa

SEQ ID NO: 28 - DNA, 20 bp, Artificial Sequence
  Synthetic polynucleotide
tcggctagggttagggtttt

SEQ ID NO: 29 - DNA, 20 bp, Artificial Sequence
  Synthetic polynucleotide
agctggttcttcgacattcc

SEQ ID NO: 30 - DNA, 23 bp, Artificial Sequence
  Synthetic polynucleotide
aaactcaaataaaacccctgctc

SEQ ID NO: 31 - DNA, 20 bp, Artificial Sequence
  Synthetic polynucleotide
gttttgcgagctcattgtca

SEQ ID NO: 32 - DNA, 21 bp, Artificial Sequence
  Synthetic polynucleotide
tgtgtgtggtctgtaggcact

SEQ ID NO: 33 - DNA, 24 bp, Artificial Sequence
  Synthetic polynucleotide
tgtatgtacacacggagagagaga

SEQ ID NO: 34 - DNA, 20 bp, Artificial Sequence
  Synthetic polynucleotide
tgaggggaagaggtttgatg

SEQ ID NO: 35 - DNA, 24 bp, Artificial Sequence
  Synthetic polynucleotide
gacccacaaacaataacctatctc

SEQ ID NO: 36 - DNA, 19 bp, Artificial Sequence
  Synthetic polynucleotide
tgggcatcatccaggtcta

SEQ ID NO: 37 - DNA, 21 bp, Artificial Sequence
  Synthetic polynucleotide
attttccgcagataatacagg

SEQ ID NO: 38 - DNA, 25 bp, Artificial Sequence
  Synthetic polynucleotide
gtttccagatctgcctccatataat

SEQ ID NO: 39 - DNA, 18 bp, Artificial Sequence
  Synthetic polynucleotide
aggcatcagttcatccaa

SEQ ID NO: 40 - DNA, 21 bp, Artificial Sequence
  Synthetic polynucleotide
ggaaggtgagagaaatcaagt

SEQ ID NO: 41 - DNA, 18 bp, Artificial Sequence
  Synthetic polynucleotide
aaggcggcactcgctcac

SEQ ID NO: 42 - DNA, 21 bp, Artificial Sequence
  Synthetic polynucleotide
gaacaactgaagacagcaaag

SEQ ID NO: 43 - DNA, 30 bp, Artificial Sequence
  Synthetic polynucleotide
agaactccgactaataatcctaacccttgc

SEQ ID NO: 44 - DNA, 28 bp, Artificial Sequence
  Synthetic polynucleotide
gagggagcaagtcaaagttgagaagaaa

SEQ ID NO: 45 - DNA, 29 bp, Artificial Sequence
  Synthetic polynucleotide
tggaggaagttttgaatggtagtagagga

SEQ ID NO: 46 - DNA, 28 bp, Artificial Sequence
  Synthetic polynucleotide
aaagcaactcatagctgaagtccaatca

SEQ ID NO: 47 - DNA, 20 bp, Artificial Sequence
  Synthetic polynucleotide
tggcgttgcaacatattctc

SEQ ID NO: 48 - DNA, 25 bp, Artificial Sequence
  Synthetic polynucleotide
gccatctttagaaagttcgatacag